(19) United States
(12) Patent Application Publication
(10) Pub. No.: US 2006/0178551 A1
(43) Pub. Date: Aug. 10, 2006

(54) Securement system for a heart actuation device

(76) Inventor: David Boyd Melvin, Loveland, OH (US)

Correspondence Address: WOOD, HERRON & EVANS, LLP, 2700 CAREW TOWER, 441 VINE STREET, CINCINNATI, OH, 45202, US

(21) Appl. No.: 11/299,197

(22) Filed: Dec. 8, 2005

Related U.S. Application Data
(63) Continuation of application No. PCT/US04/18299, filed on Jun. 9, 2004.
(63) Continuation-in-part of application No. 10/667,877, filed on Sep. 22, 2003.
(60) Provisional application No. 60/477,078, filed on Jun. 9, 2003.

(51) Int. Cl. A61N 1/362

(52) U.S. Cl. 600/16

(57) ABSTRACT

The invention consists of means (1, 64, 81) of securing a powered active cyclically deforming jacket (13) that surrounds the outer or epicardial surface (12) of at least one chamber of the natural heart, as well as auxiliary components. Internal components suspended from the jacket (13) by heart wall-penetrating cords complete a restraining harness over the entire 3-dimensional boundary of the chamber or chambers. The embodiments of the invention also teach means (1, 64, 81) that are suitable to achieve secure fixation with limited operative exposure such as would be available in minimally invasive approaches, and means (89, 90, 91) by which later cardiac operation such as coronary bypass can be facilitated despite presence of the heart actuation device.

This application is a continuation of PCT/US2004/018299 filed on Jun. 9, 2004, which claims priority of U.S. Provisional Patent Application No. 60/477,078, filed Jun. 9, 2003. The disclosure of each priority application is hereby incorporated by reference herein in its entirety.

This application is a Continuation-In-Part application of U.S. patent application Ser. No. 10/667,877, filed Sep. 22, 2003 and entitled “Basal Mounting Cushion Frame Component to Facilitate Extrinsic Heart Wall Actuation” which application is hereby incorporated by reference in its entirety.

### FIELD OF THE INVENTION

This invention relates generally to assisting the natural heart in operation and, more specifically, to components to assist in actuating one or more walls of the natural heart.

### BACKGROUND OF THE INVENTION

The human circulatory system is critical for survival and systematically provides nutrients and oxygen as well as removing harmful waste products from all parts of the body. The heart is a critical component of the circulatory system in that it provides pumping power. Generally the right side of the heart receives blood from the ‘systemic circulation’ (all the body except the lungs) and pumps it into the ‘pulmonary circulation’ (lungs), whereas the left side of the heart receives blood from the lungs and pumps it back into the systemic circulation. Each side comprises an inflow or collecting chamber with a thin muscular wall, its ‘atrium’ and a thicker, more powerful muscular pumping chamber, its ‘ventricle’, which alters volume cyclically due to contraction and relaxation of the muscles in its walls. One-way valves are positioned in the passageway between the left and right atrium and the corresponding ventricle, and between each ventricle and the large arteries that conduct blood into the systemic or pulmonary circulation, respectively. Because of this arrangement, each atrium may gently contract, causing blood to flow across the ‘atrioventricular’ valve into the ventricle, with that valve then closing to prevent return. Similarly, each ventricle may then forcefully contract, causing blood to flow across the outflow valves into the systemic or pulmonary circulation. A physical ailment or condition which compromises the effective muscular contraction in the walls of one or more chambers of the heart can therefore be particularly critical and may result in a condition which must be medically remedied if the person is to long survive.

More specifically, the muscle of the heart may degrade for various reasons to a point where the heart can no longer provide sufficient circulation of blood to maintain the health of a person at an acceptable level. In fact, the heart may degrade to the point of failure and not been be able to sustain life. To address the problem of a failing natural heart, solutions are offered to maintain the circulation. Some of these solutions involve replacing the heart. Some involve assisting it with mechanical devices. Some are directed to maintain operation of the existing heart.

The heart may be removed and replaced with either a mechanical device (a total artificial heart) or a natural heart from another human or an animal (heart transplant). Artificial heart use has been complicated by consequences of blood clots forming on the internal lining. The most serious consequence is a breaking loose of such clots, which are then propelled into various parts of the circulation. In the event of such a clot being propelled into the brain, a disabling or fatal stroke may result. While human heart transplantation is limited by rejection, a response of the body's immune system, this may usually be controlled by medications to the degree that half of all recipients survive at least 10 years, generally with acceptable health and function. However a more serious limitation is numbers of available donors. These are usually accidental death victims whose hearts maintain function despite brain death. Currently these are available for less than 1 to 2 percent of potential beneficiaries (about 2000 per year in the United States for over 200,000 people dying of heart failure annually in the same country, for example).

The heart may be assisted by mechanical auxiliary pumps. These are of three general types: counterpulsators, pulsatile assist systems, and nonpulsatile assist systems. Counterpulsators such as intraaortic balloon pump cyclically remove or displace blood from the arterial system in synchrony with the natural heart's beat and, without valves, may perform substantial work for a weakened heart. Pulsatile assist systems (ventricular assist devices) are similar to artificial hearts except that they are used in addition to one or both sides of the heart rather than instead of the heart. They receive blood from either the atrium or ventricle on one side of the circulation and pump it into that side's arterial system, relieving the ventricle of part of its volume load, pressure load, or both. They consist of a blood chamber with at least partial wall flexibility, inflow and outflow valves, and some means, usually pneumatic, hydraulic, or electric, by which the wall may be moved and volume altered to pump blood. Nonpulsatile assist systems are rotary pumps, either centrifugal, axial flow, or a combination, that similarly pump blood in a steady flow from atrium or ventricle into circulatory systems. All of these mechanical pumps have extensive non-living material surfaces that contact blood. The complications of blood clotting with stroke or other serious aftermaths described with artificial hearts also occur with these mechanical auxiliary pumps.

Because of the severe shortage of human donor hearts for transplant, unsolved immunologic problems of animal donor hearts for transplants and prevalence of serious complications of artificial blood-contacting surfaces of both artificial hearts and auxiliary pumps, means of aiding the actuation of the natural heart walls have been attempted. Both skeletal muscle wraps (‘cardiomyoplasty’) and mechanical compression devices (‘mechanical ventricular actuation’) have been used. In either approach, the external wall surfaces of the heart are compressed and the heart volume altered, thereby pumping blood out of the chambers. Muscle wraps are limited by available space relative to muscle mass required for power, as well as by intrinsic stiffness that compromises re-filling between beats. Both muscle wraps and mechanical compression devices are limited by inability to effectively restrict volume and pressure delivery to one chamber of the heart. This chamber restriction is important because the two sides of the circulation require far different pressures for acceptable function (usually the systemic pressure is 3 to 5 times as high as is the pulmonary pressure). Compressive patterns of either muscle wraps or mechanical devices may also distort heart valves, which can lead to valve leakage.

Therefore, to be effective and safe, mechanical pumping of a person's existing heart, such as through mechanical compression of the ventricles or some other action thereon, must address these issues and concerns in order to effectively and safely pump blood. Specifically, the weakened ventricle or ventricles must rapidly and passively refill between beats at low physiologic pressures, and the valve function must be physiologically adequately. The blood flow to the heart muscle must not be impaired by the mechanical device. Still further, the left and right ventricular pressure independence must be maintained within the heart.

Internal stabilizing components to complete the three-dimensional control of a chambers' boundaries, which components are suspended through the substance of heart walls from the external (to the heart) actuating mechanism should be a useful adjunct. These provide a means to facilitate the precise control of actuation—determining the prescribed pattern and distribution needed to (1) prevent valvular distortion, (2) avoid myocardial blood flow compromise, (3) provide a type of shape alteration of the actuated chamber at end-actuation which will facilitate passive refilling during shape restoration, and (4) ensure relative independence of pressure in the various chambers.

Specifically, U.S. Pat. No. 5,957,977, which is incorporated herein by reference in its entirety, discloses an actuation system for the natural heart utilizing internal and external support structures. That patents provides an internal and external framework mounted internally and externally with respect to the natural heart, and an actuator device or activator mounted to the framework for providing cyclical forces to deform one or more walls of the heart, such as the left ventricular free wall. The invention of U.S. patent application Ser. No. 09/850,554, which has issued as U.S. Pat. No. 6,592,619, further adds to the art of U.S. Pat. No. 5,957,977 and that patent is also incorporated herein by reference in its entirety. The application specifically sets forth various embodiments of activator or actuator devices that are suitable for deforming the heart walls and supplementing and/or providing the pumping function for the natural heart.

While the actuation systems of those patents provide a desirable actuation of the natural heart, it is further desirable to improve upon security and safety of means of attaching hardware to the heart.

It is still further desirable to modify devices so that they are suitable for placement through small operations (minimally invasive operative access). It is yet still further desirable to provide devices and methods that make possible safe access to the heart for future operations, such as coronary bypass.

### BRIEF DESCRIPTION OF DRAWINGS

FIG. 1 shows ‘bolsters’ useful for securing hardware to the heart.

FIG. 2 illustrates balled tips to be placed on needles to lessen trauma in heart muscle penetration.

FIG. 3 shows use of a ball-tipped needle to place a suture into myocardium.

FIG. 4 shows continuing advancement of the needle and suture of FIG. 3.

FIG. 5 shows a left-sided ‘active heart jacket’ as an example of hardware that may be placed on the surface of the heart using sutures placed by the methods of FIGS. 3 and 4.

FIG. 6 is a cross-section showing one of the bolsters of FIG. 1 that has been placed by the methods of FIGS. 3-5 to secure epicardial hardware to the region of the anterior interventricular septum.

FIG. 7 is a perspective of placement of a bolstered suture in the anterior mural-septal angle of the right ventricular outflow tract through one of two small right ventriculotomies.

FIG. 8 is a right ventricular cut-away view of a septal supporting splint created of webs of prosthetic material using the techniques of FIGS. 3-7.

FIG. 9 is a close up view of the splint in the region of the tricuspid valve and its chordal support, both soon after placement and after a period of healing and endothelialization.

FIG. 10 is a laminar tension-indicator surface, first in exploded view and then successively indicating acceptable and excessive tension.

FIG. 11 shows an elastic loop tension-indicator device.

FIG. 12 shows a spring-loaded pinch clamp to secure sutures which have penetrated tissue, such as the heart wall, at a desired tension without tying, and device which releases the pinch clamp at a preset and adjustable suture tension, with or without cutting.

FIG. 13 is an epicardial (heart outer surface) button which may be fixed across the heart wall to an endocardial (heart inner-surface) bolster at a desired tension by the methods of FIGS. 10-12 or other means, and subsequently used as a fixation point for additional, functional, hardware and devices.

FIG. 14 shows a curved stylus configured for placement of the strands of a septal splint, such as shown in FIGS. 8 and 9, without necessarily opening the heart chamber, as well as examples of paths along which it may be placed.

FIG. 15 shows one method of utilizing the stylus of FIG. 14, in which an enclosing tubular strand is pushed across the heart chamber and the stylus withdrawn.

FIG. 16 shows another method of utilizing the stylus of FIG. 14, in which it is advanced without a strand, and the strand is attached to the protruding end, which is then pulled through the heart along the surface of the septum.

FIG. 17 is an umbrella like bolster, which may be advanced over a strand from outside to in and opened within the heart as well as a pusher tool to facilitate use.

FIG. 18 is a variation of the umbrella bolster that is placed on a penetrating suture or cord, which is subsequently allowed to retract through the heart wall, at which point the bolster may open.

FIG. 19 shows a region of a heart jacket that has been ‘windowed’ to access the site of a coronary artery, such as for bypass, in which there is an easily separable plane between a thin flexible layer, which remains adhered to the heart surface, and the thicker, stiffer, heart jacket body, which does not remain adhered.

FIG. 20 shows devices and methods for performing and safeguarding a coronary bypass anastamosis under a heart jacket.

### DETAILED DESCRIPTION OF THE INVENTION

Part Numbers
1. bolsters
2. soft porous bolster surface
3. suture pre-fixed to bolster
4. needle on suture which is prefixed to bolster
5. polymer loop as and example of a means of securing septal-splint stringing material
6. ball tips to be placed, permanently or temporarily, on the point of needles to facilitate passage through myocardium.
7. points of needles onto which ball tips are placed
8. a length of hypodermic tubing which may facilitate intraoperative placement of balled tips on needles
9. inner surface of heart or ‘endocardium’
10. right ventricle
11. left ventricle
12. outer surface of heart or ‘epicardium’
13. active cardiac jacket
14. aortic sleeve
15. sutures exiting from base of right side septal support
16. sutures exiting from lateral margins of right side septal support
17. a patch repair for atrial separation
18. tricuspid valve
19. mitral valve
20. aortic root
21. transmural cords
22. stringing material
23. ventriculotomies
24. right ventricular free wall
25. net-like septal support
26. chordae tendinae
27. trabeculae
28. papillary muscles
29. surface layer of multilaminate pressure indicator
30. second layer of multilaminate pressure indicator
31. third layer of multilaminate pressure indicator
32. fourth layer of multilaminate pressure indicator
33. fifth layer of multilaminate pressure indicator
34. region indicating desirable degree of compression
35. region indicating excessive degree of compression
36. elastic loop
37. stretch indicator grid
38. region indicating inadequate tension
39. region indicating excessive tension
40. knot, single throw, loose
41. knot, single throw, snug
42. pinch clamp
43. jaws of pinch clamp
44. ridges on jaws of pinch clamp
45. spring of pinch clamp
46. trip pin for pinch clamp
47. pinch clamp applicator
48. applicator handle
49. applicator body
50. applicator stage
51. cavity
52. upper margin of cavity
53. lower margin of cavity
54. hole for suture or cord
55. fixed pulley A
56. sliding pulley
57. fixed pulley B
58. slot
59. piston
60. piston channel
61. piston socket
62. piston compression spring
63. pin socket
64. button
65. button core
66. stylus
67. stylus tip
68. stylus grasping tab
69. stylus aspiration port
70. stylus aspiration tube
71. stylus, and cord, path example 1
72. stylus, and cord, path example 2
73. stylus, and cord, path example 3
74. stylus, and cord, path example 4
75. stylus, and cord, path example 5
76. strand
77. ventricular septum
78. detachable ball tip for stylus
79. initial stylus entrance point
80. initial stylus exit point
81. disk section of a ‘umbrella’ type externally positioned bolster
82. stays
83. tubular section
84. retaining cords
85. stop-ring
86. filler core
87. pusher tool
88. suture
89. thin polymer mesh intended to adhere to epicardium as an inner liner for jacket
90. readily separable means of adhering mesh to jacket
91. section of jacket removed for coronary access and replaced after bypass anastamosis
92. heart
93. coronary artery
94. bypass graft to coronary artery
95. preformed epicardial cushions to protect anastamosis

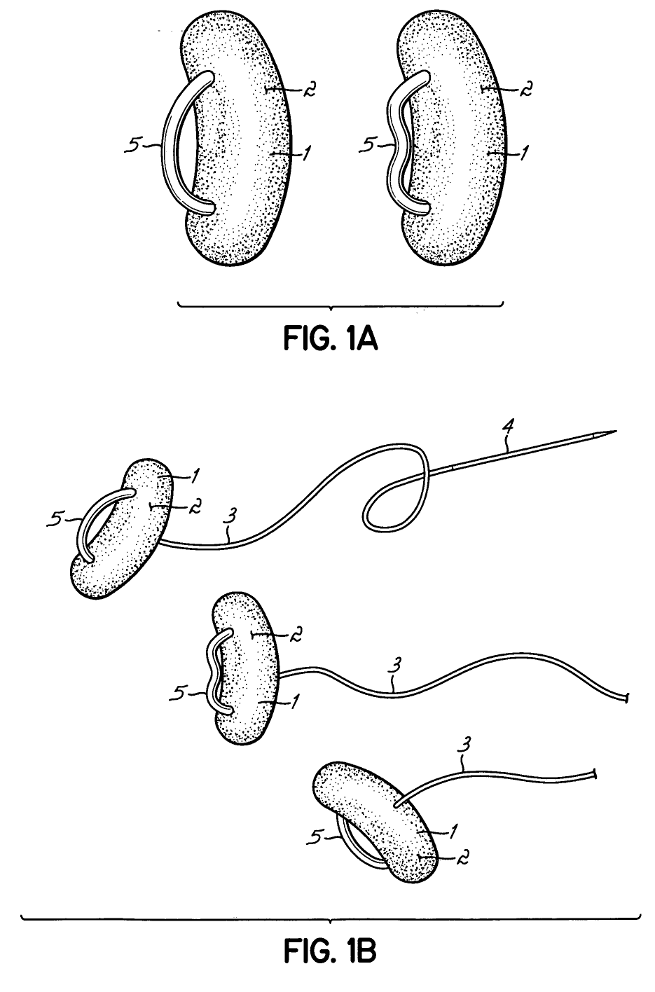

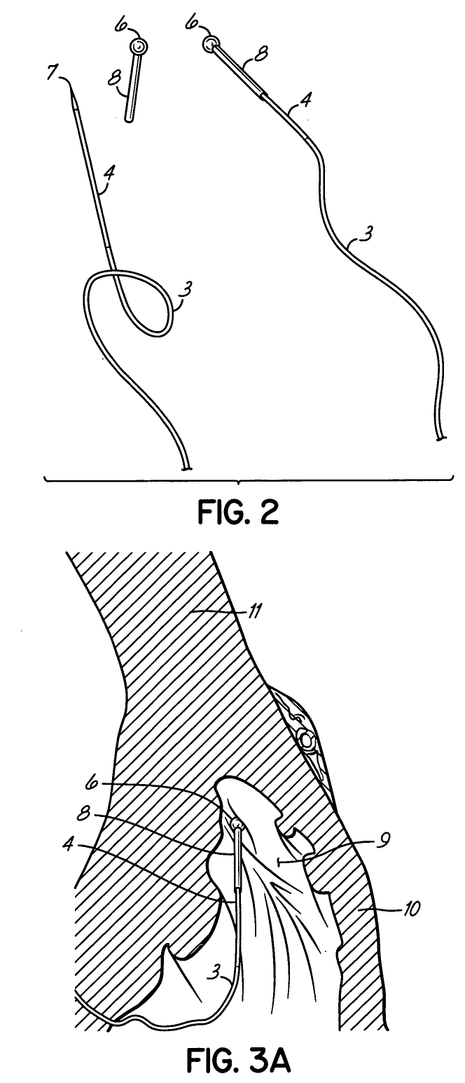

A series of ‘bolsters’ [1], soft, and smooth contoured, shown in FIG. 1a, is configured to be held against the endocardium. They are surfaced [2] with a soft porous material. The bolsters may have at least one suture [3], as shown in FIG. 1b, preferably with a swaged-on straight taper-tipped needle [4], extending from one surface. The bolsters may be equipped with means of securing strands of the stringing material, generally one or more loops [5] of polymer or metal placed approximately opposite the fixing suture. Bolsters are configured to be held against the endocardium at or near the margin of one side of the interventricular septum, at or near the intersection of that septum and the ventricular free wall and at or near the intersection of that septum and the valve annuli of that ventricle. The bolsters may be rounded end cylinders, kidney-bean shaped. The bolsters may be made of a soft elastomeric material, such as low-durometer silicone or polyurethane, and are surfaced [2] with a soft porous material such as a polyester knit or velour.

Balled tips [6] either may be pre-fixed on or may be placed on point [7] of needles to facilitate their safe passage through myocardium, lessening the likelihood of damage to vessels or other structures. The ball tips may be mounted on a cylindrical segment of metal hypodermic tubing [8] that may be placed on the needle tip before or at operation and removed when required.

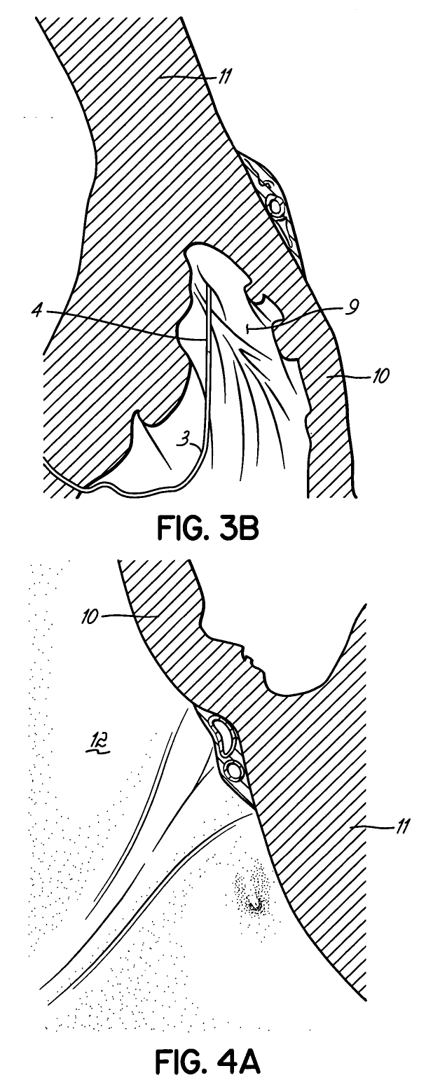

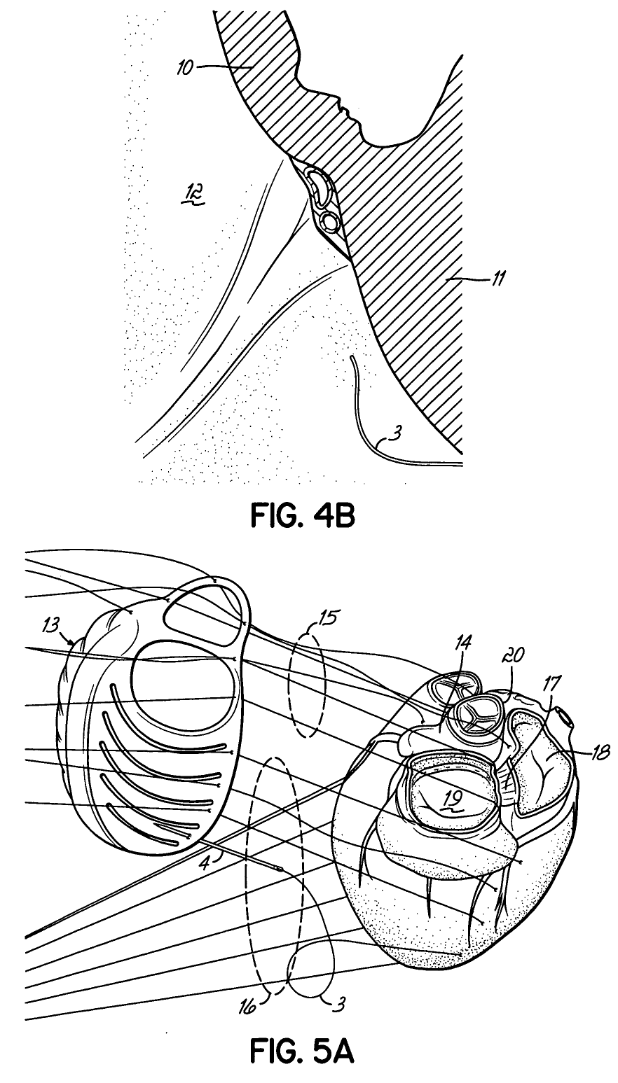

A method of placement of bolsters comprises pressing the ball-protected needle tip against the inner lining tissue of the heart (the ‘endocardium’) [9], optionally with a preparatory ‘knick’ in the local endocardium, and pressing, optionally with a gentle vibratory motion, as it advances in and through the heart wall, generally at or near the junction of the right ventricle [10] and the left ventricle [11], as in FIG. 3a and FIG. 3b, optionally using a small ‘knick’ in the opposite surface to facilitate exit through the outer surface of the heart (the ‘epicardium’) [12] as in FIG. 4a and FIG. 4b.

An optional elongated needle (similar configuration, several times as long) allows the method of placement to be modified for access other than ventricular incision, variably configured for use through a ventricular free wall puncture, an atrial puncture, or a venous puncture, with use of fluoroscopy or other imaging guidance as required.

The provisions described here for placement may be augmented by preliminary or coincident visualization by epicardial ultrasound or other means of identifying vulnerable structures such as coronary arteries.

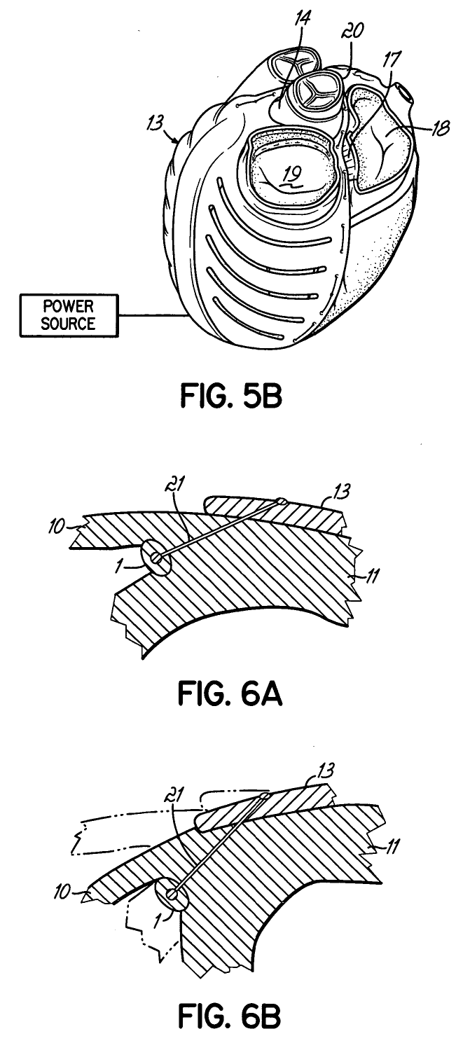

In the preferred method of seating the one particular wall actuating component, an ‘active jacket’ [13], the needles of the completed circumferential row of bolsters, exiting the ventricular or atrial external surfaces, are advanced through the substance of the jacket near its margin (FIG. 5a), following by lowering the jacket into place and tying (FIG. 5b) in a manner used by and familiar to cardiac surgeons for sewing and seating a prosthetic heart valve. Generally a cushioned aortic sleeve [14], surrounding the base of the aorta and covering the proximal coronary arteries, will have been placed prior to this step. Sutures exiting from base of right side septal support [15] and sutures exiting from lateral margins of right side septal support [16] will be present and a patch repair [17] for atrial separation will already be present. Anterior and to the right of the circumferential set of exiting cords or sutures will be the right ventricle [10] and tricuspid valve [18], and to the right of the cords will be the left atrium enclosing mitral valve [19], the aortic root [20], and the left ventricle [11]. As illustrated I FIG. 5B, the jacket, which includes an actuator mechanism, is coupled to a power source for actuation. One version of such a jacket is disclosed in the patent application entitled “Deforming Jacket for a Heart Actuation Device, filed on Jun. 9, 2004 and discussed further hereinbelow.

This creates a series of cords [21] across the myocardium such as illustrated in FIG. 6. These cords become taut with tensile loading and transfer force between the jacket [13] and the bolsters [1] without delivering force to myocardium beyond that of maintaining its own position relative to the assembly. In a modified similar method, the sutures are mechanically fixed to prepared receiving elements in the jacket near its margins.

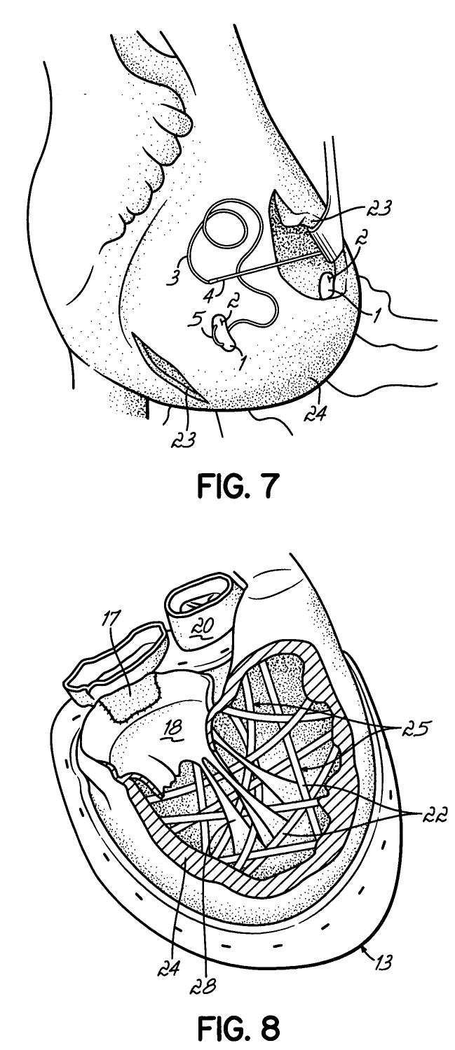

A ‘stringing’ material [22], such as braided polyester suture, ePTFE (expanded polytetraflurethylene) or braided polyester ribbon, is pre-prepared for use, in one or more segments, to link bolsters across the septum, thus creating a ‘snow-shoe-like’ network so that the septum is supported from the bolsters and thus indirectly by the jacket margins to which the bolsters have been anchored across the heart walls. Access for stringing is generally direct vision through one or more small (2 to 4 cm long) incisions (‘ventriculotomies’) [23] in the right ventricular free wall [24] as in FIG. 7.

As shown in FIG. 8, method of ‘stringing’ the net-like septal support [25] comprises placing strands between, behind, or through, chordae tendinae [26], trabeculae [27], papillary muscles [28] or other structures which require protection to ensure continued inflow valve function. A nonlimiting example of the completed support [25] is shown in a cutaway view of the septum with the right ventricular free wall [24] artificially removed for illustration.

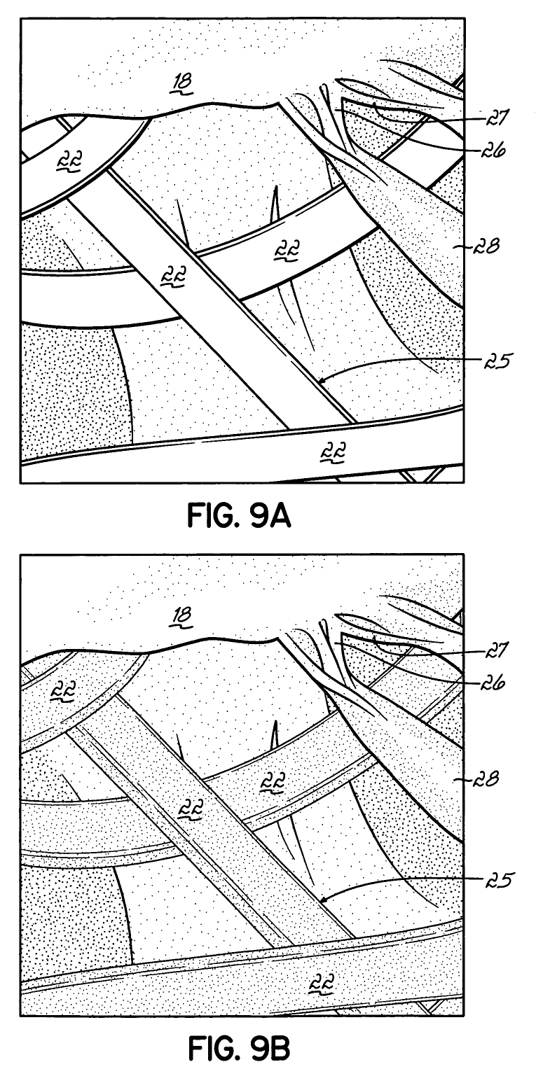

FIG. 9 shows a closer view after such stringing. FIG. 9a is a close-up view of a section of the grid or net support [25] so created freshly after placement and FIG. 9b is the same view as it would appear after endothelialization, the process in which the native heart lining tissue tends to grow over and anchor to any porous material that is not excessively flexing.

An indicator device may be used with this method, calibrating tightness of tied sutures joining bolsters through myocardium to jacket [13]. Such an indication is disclosed in a related patent application entitled “Deforming Jacket for a Heart Actuation Device” and filed on Jun. 9, 2004 as a PCT application, which application is incorporated herein by reference.

The indicator device may function as a surface tension indicator to allow control of suture tying tightness in which a laminated structure, with alternate laminae translucent and either textured or colored, in such a way that a visible change signals achievement of adequate tightness for control of bleeding. This indicator system may be constructed in such a way that a visible change signals achievement of excessive tightness that, if not reversed, could risk tissue damage.

A tension-calibrating device for fixing sutures may be an adaptation of ratcheted tension-control fastening devices familiar to those in both cardiac surgery and engineering design (e.g. ‘snap-band guns’) configured to work with sutures after penetrating from bolsters and regulate tension at which sutures are mechanically fixed. The ratcheting and tension limiting features of such devices may be adapted and incorporated into prefixed openings in the margins of the jacket into which the sutures may be inserted. Because of the risks of tying or fixing these jacket-fixing sutures or cords either excessively loose (i.e., bleeding from the heart surface) or excessively tight (i.e., reduction of blood flow to the heart tissue supplied by any coronary arteries traversing the region), means of measuring or otherwise controlling the tension with which these sutures or cords are placed may be useful. The teaching of this invention includes examples of such means:

An indicator device may be used for calibrating tightness of tied sutures joining internal structures, such as bolsters or frame struts, through myocardium to jacket. The indicator device may function as a surface tension indicator to allow control of suture tying tightness in which a laminated structure, with alternate laminae translucent and either textured or colored, in such a way that a visible change signals achievement of adequate tightness for control of bleeding. This indicator system may be constructed in such a way that a visible change signals achievement of excessive tightness that, if not reversed, could risk tissue damage.

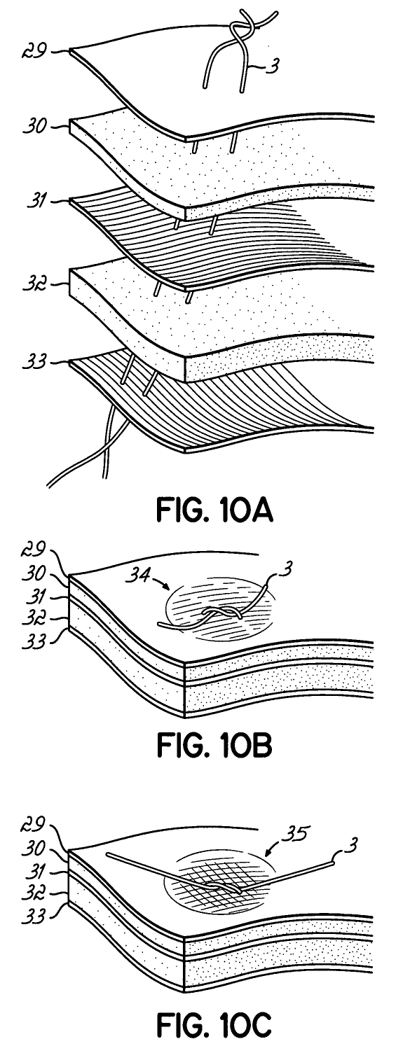

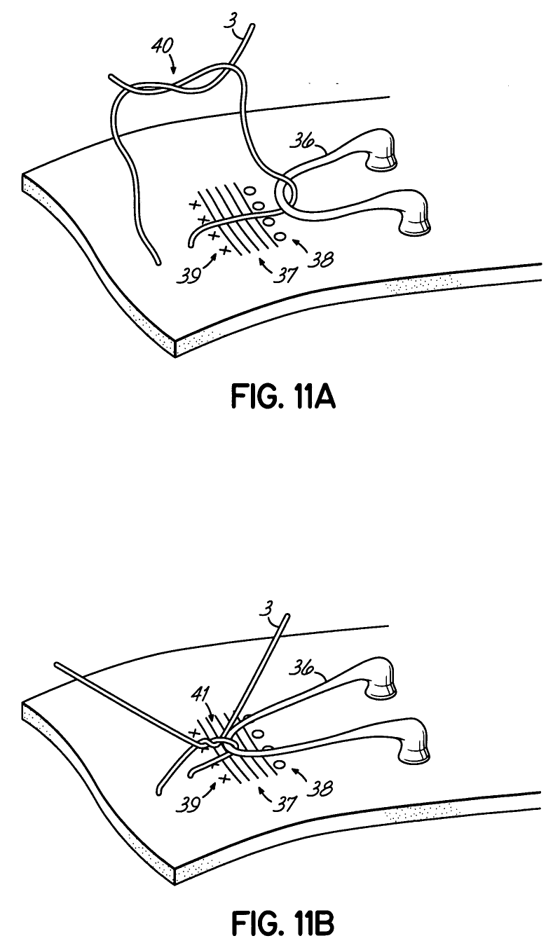

A specific example of such a system follows, as illustrated in FIGS. 10a, 10b, and 10c. The jacket may be equipped on part or all of its outer surface with a translucent layered composition structured so that tying of a penetrating suture [3] results in a local color or other visible change that is at least semi-quantitatively related to the tightness of tying and thus to the compressive stress being imposed on the underlying heart surface. A nonlimiting example is a laminar structure in which the first, third and fifth layers [parts 29, 31, and 33] are transparent and either colored yellow, blue, and red, respectively, or having none, left-to-right cross-hatches, and up-to-down crosshatches, respectively, with the first layer being the outer surface [29]. Each of these layers are of a thin elastomer with the appropriate pigment or lining added and preferably reinforced with a fine polymer fiber mesh to enhance tear resistance. Between the first and third layers, the second layer 2 [part 31] is a very thin layer of extremely low durometer (e.g., an order of magnitude softer than the pigmented or cross-hatched layers) clear elastomer, and between the third and fifth layers, the fourth layer 4 [part 32] is similar to the second layer except either thicker or firmer—and thus less easily compressed. The compliant second [30] and fourth [32] layers may contain suspended colloidal particles or gas bubbles so as to increase light diffusion and thus decrease transparency when not substantially compressed.

This structure will provide an indicator of the compressive force of a tied penetrating suture loop. The mechanism is that a certain degree of compressive stress will cause sufficient thinning of the second layer [30] that the initially visible yellow surface layer [29] color becomes green as it becomes compressed against the blue layer [31], and then brownish gray as the three layers are all closely compressed. In the lined variant, thinning of the second layer [30] with pressure causes the non-lined appearance of the surface layer [29] to transform locally to the left-to-right crosshatching as layer 3 becomes visible through it, and by a similar process at higher tying pressure then transforms to a grid pattern as the fifth layer [33] also becomes visible from the surface in the immediate vicinity of the suture being tightened. Thus a desirably compressed region [34] and/or any excessively compressed region [35] will be readily recognizable. As will be apparent, for either variant construction parameters may be selected such that the yellow to green (or clear to lined) transition occurs at a compressive stress which in the underlying tissue is expected to arrest bleeding, while the green to brownish-gray (or lined to gridded) occurs at a stress somewhat lower than one at which tissue ischemia is risked. This may be calibrated based on experimental assessment to a level where tying ‘tight enough but not too tight’ is readily achieved by visual guidance.

FIG. 10a is an ‘exploded’ view of laminar construction as described in the prior paragraph.

FIG. 10b illustrates the first transition where single direction cross hatch marks are visible, representing achievement of the ‘safe and necessary’ pressure, whereas the grid cross hatching in FIG. 10c indicates the point of excessive compressive stress—suggesting the suture be loosened.

An alternative means of calibrating suture tying tightness while securing the jacket [13] is the tensile elastic element such as a spring of various configurations or a loop [36] of elastomeric polymer such as shown in the nonlimiting example of FIGS. 11a and b. This is complemented with visible indicators of the elastic element's extension, and thus tension placed upon it. These indicators may be ridges, grooves, color bands, dashes [37] or other marks and may be labeled with numbers, letters, and so forth. Special markings such as the ‘0's’ [38] may indicate inadequate tension and the ‘X's’ [39] (both in FIG. 11a) may indicate excessive tension. The tensile elastic element is based at a short distance from the site of suture penetration and looped by the penetrating suture ends before tying. Deformation may be gauged by marks on the jacket surface to be too loose, as the beginning knot [40] in FIG. 11a or in a range deemed safe [41] as in FIG. 11b and the knot completed. To avoid cyclic elastic deformation during actuation, which may be undesirable, the established position may be made permanent by any commonly used fixation technique such as another suture, a staple, or a brad, or a combination of these or other techniques and devices to anchor the tied cord or suture to the substance of the jacket at the determined location.

Yet another alternative is use of a tension-calibrating device for fixing sutures which is an adaptation of ratcheted tension-control fastening devices familiar to those in both cardiac surgery and engineering design (e.g. ‘snap-band guns’) configured to work with sutures after penetrating from bolsters and regulate tension at which sutures are mechanically fixed.

The ratcheting and tension limiting features of such devices may be either adapted and incorporated into prefixed openings in the margins of the jacket into which the sutures may be inserted or configured to be used with sutures penetrating the jacket's substance by needles or by other means.

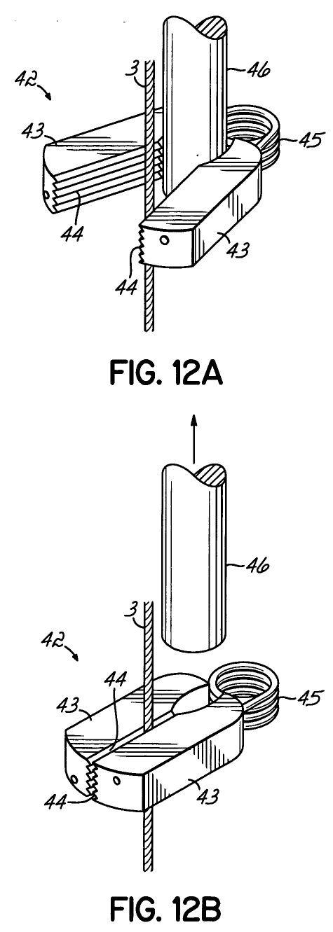

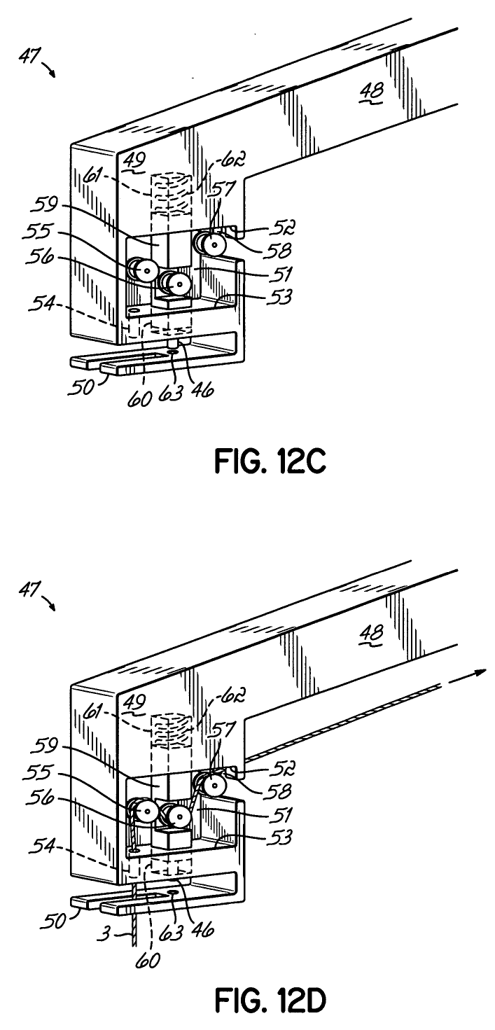

Yet another means of tension control is a miniature but very secure suture clamp of the type shown in FIG. 12a through 12d. A nonlimiting example of a spring-loaded pinch clamp [42] for this purpose is shown in FIG. 12a and 12b. The two jaws [43], preferably of a polymer such as a thermoplastic are mounted on the arms of a helical spring [45], preferably of stainless steel; resting position of the spring is such that the jaws [43], generally with interlocking ridges [44] are held together. The spring [45] is sufficiently strong that when jaws are allowed to approximate, any cord [3] or suture of diameter 0.2 to 1.5 mm will be grasped tightly so that a force in the range of 45 to 90 N (˜10 to 20 lb) is required for slippage. The clamp is configured to be held open until released, for example by the withdrawal of a pin [46], generally by means of an applicator that responds to cord tension, such as that of FIGS. 12c and 12d.

Such a clamp is temporarily mounted on an applicator. In the nonlimiting example shown here the applicator [47] is cast or machined from a rigid material, preferably a solid polymer such as polyethylene or polypropylene. It has a handle [48] contiguous with a body [49], with the body connected to a stage [50]. In the body there is a cavity [51] open on one side; the cavity has an upper margin [52] and a lower margin [53]. In use, the clamp is positioned on a stage [50] and held open by an instrument while pin [46] is lifted by another instrument. In the setting of a surgical operation these maneuvers could be readily done using hemostats or a hemostat and a forceps, respectively. With a similar instrument, the clamp is moved into position so that the pin [46], until released, holds the clamp open as shown in FIG. 12a. Then the cord or suture [3], at its exit from the heart surface, is threaded through hole [54], over fixed pulley [55], under sliding pulley [56], over fixed pulley [57] and through slot [58]. Sliding pulley [56] is mounted on a piston [59] that moves in a channel [60] in the lower margin [53] and an aligned socket [61] in the upper margin. The base of socket [61] contains a compression spring [62]. Pin [46] is mounted on the lower end (as drawn) of piston [59]. The channel [60] is of sufficient size to accommodate the piston [59] in the majority of its length but only the pin [46] for a small portion, generally about ¼, of its length nearest the stage 10. Compression spring [62] is configured such that piston [59] is pushed down by it such that the piston's end rest against this constriction in the channel. Pin [46] protrudes through the channel for sufficient length to touch or almost touch the stage, and may seat within pin socket [63] in the stage.

After loading, traction may be exerted on the cord [3] by the operator. This is generally done manually, holding the handle 48 in the nondominant hand and the cord [3] in the dominant hand. It is important that the wrist of the handle-holding hand be relaxed so that the stage [50] rests freely on the heart surface [12] or jacket [13] surface. The tension generated in the cord exerts an upward force on sliding pulley [56] and thus on piston [59] and compression spring [62]. The spring constant of spring [62] and its longest permitted configuration (i.e., its length when piston is resting on the channel constriction) are selected so that the spring will be compressed further upon achievement of a targeted minimum tension in the cord [3] and be compressed sufficient to withdraw the pin [46] completely into the channel [60] with tension no greater than ˜120% of this value. At that point, jaws [43] are released and grasp cord [3]. Upon sensing this, generally by means of hearing a clicking noise, the operator releases the cord, removes it from the applicator, and cuts excessive length. It will be apparent that simple additions to the design would permit an optional cutting blade to automatically divide the cord above the clamp immediately after closure.

Note that effective tension is that between cord and applicator. If applicator is held with any substantial force away from the heart (i.e., if it does not rest freely on the heart surface) the clamp could be released with tension in the cord at heart exit lesser than the minimum desired. Thus an alternative design omits stage [50]. This necessitates care in positioning the clamp so that its grip on pin [46] alone assures proper angulation as it is brought into contact with the heart surface and tension commenced. Note also in a no-stage applicator, the minimum tension selected may be modestly lower than in an applicator that does have a stage. This is because applicator removal in a staged applicator will add the thickness of the stage (generally 1-2 mm) to intramyocardial cord length, lessening tension slightl.

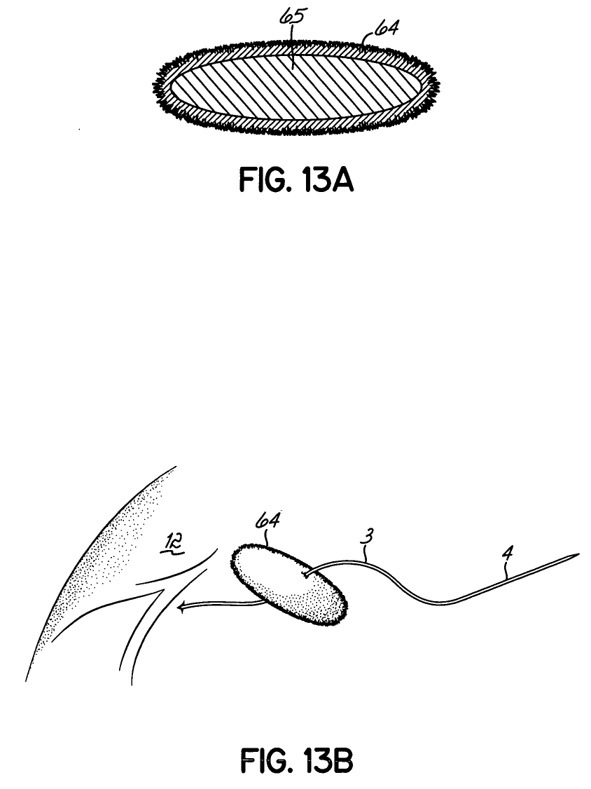

A means of potentially improving access to and visibility of individual transventricular fixation points is illustrated in FIGS. 13a and 13b. A ‘button’ [64], which may be velour covered and has a core [65] which may be of either a soft material such as an elastomer or a hard material such as a solid polymer of a metal, is initially fixed to the suture or cord [3] beyond the point that it has penetrated the epicardium [12], indirectly connecting to internal structures such as an internal bolster. Fixation to individual buttons, and tension control of the transmyocardial cords, may be accomplished by any of the means described for fixation to the jacket margin. The buttons are in turn fixed to the jacket margin by sutures, hooks, staples, or any type of mechanical fastener. This may have an advantage in allowing tensions in various cords, and thus initial pressure on their underlying tissue, to be individually controlled. FIG. 13a is a cross-section and 13b is a perspective view of a cord [3] being placed through button [64] after exiting epicardium [12].

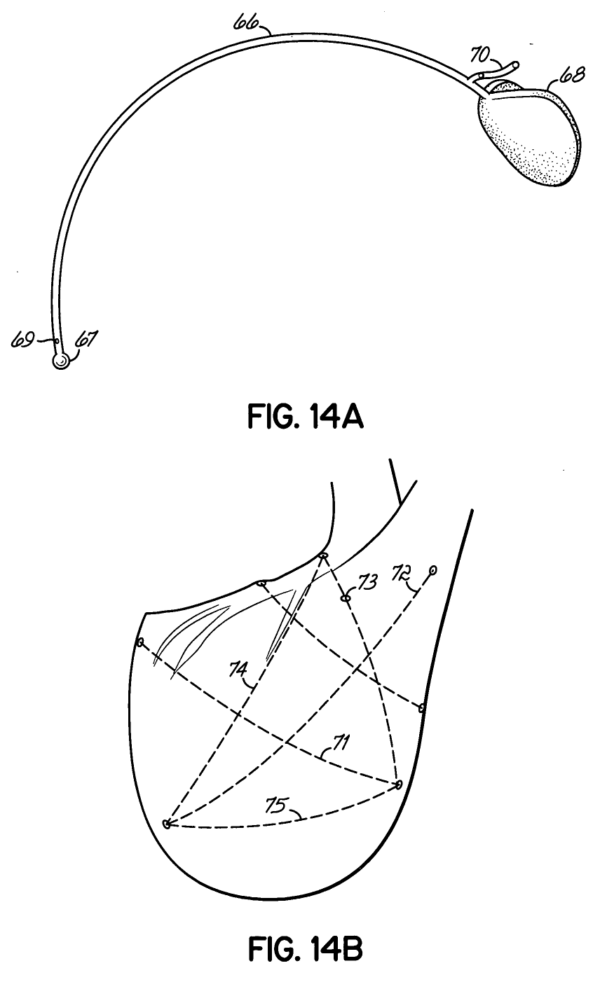

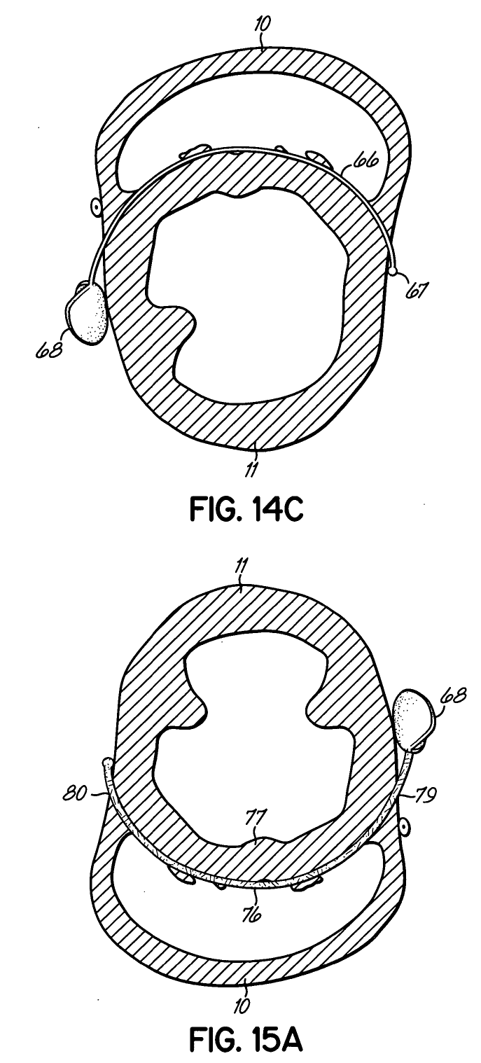

Still yet another embodiment provides devices and techniques permitting construction of a sling or net to mechanically control the ventricular septum, and to suspend that sling or net from the portion of an active jacket on the heart surface, through only punctures, without incisions in the right or left ventricle. First, styli [66] (shown in FIG. 14a), with tips [67] similar to those of the ball-tipped needles employed for bolster placement, are used. These styli are generally 8 to 12 cm (˜3.5 to ˜5 inches) long with the minimum length determined as 2 to 3 cm greater than the curvilinear distance from their proposed entrance to their proposed exit site on opposite sides of the right ventricular margins. These are curved to approximate the anatomic path intended and have grasping tabs [68] (not included in stated length) to facilitate their placement. They also preferably have an aspiration port [69] on a lateral surface near the tip connected by an aspiration channel to an aspiration tube [70] adjacent the grasping tab. In general, a variety of styli of different lengths and curvatures will be prepared to be individually chosen to approximate observed cardiac geometry.

A minimum of five needle paths, which will subsequently be cord paths, are required, as illustrated in FIG. 14b as projections on the right ventricular free wall. These paths marked externally to terminate in regions that are not within 5 mm of visible coronary vessels or veins on or near the cardiac surface, with liberal use of epicardial echocardiography probe confirmation. After a nick (for example, by a 18 gauge needle prick), the ball tip is placed an in a gentle vibratory movement advanced in the direction intended. Once in the right ventricular cavity, (FIG. 14c, a sectional view) attention is given to advancing with the ball tip [69] against the septum to minimize trapping of trabeculae or chordae. When the ball tip' bulges and pallor is visible near the exit site, location is confirmed by ultrasonic visualization, repositioning is done if needed, and the stylus passed through. Note that entrance sites, over which there may be tighter visualization and control, are chosen near the base, located where the larger and more vulnerable vessels generally present may be more easily seen directly, with exit sites nearer the apex of the heart. During advancement of the stylus, the aspiration tube may be intermittently aspirated by an assistant by means of a syringe, testing whether the aspiration port near the tip is indeed in a heart chamber and whether blood appears unsaturated as would be expected in the right ventricle. It will also be apparent that an alternative or supplement to the aspiration port, channel, and tube system may be an oximetric sensor of the type frequently used on pulmonary artery and other catheters, connected to appropriate external meters.

Entrance and exit sites are targets only. Depending on anatomy, for example, an entrance that is approximately a few millimeters removed from that outlined may be chosen due to pattern of coronary vessels—both arteries and the coronary sinus and its tributaries. Typical paths are outlined below as projections [71] to [75], corresponding to the numbers in the left column of the table] on the surface of the right ventricle [10] In the preferred technique, each stylus is passed and then withdrawn, generally by one of the two methods to be described following the table, leaving a strand of flexible porous material extending along its path and joining the entrance and the exit points.

| # | Entrance | Technique | Exit |
|---|---|---|---|
| 1 [71] | Lower margin AV fat pad | Left ventricle either beating | Anterior inter- |
| | posteriorly, below coronary | and ejecting or full to static | ventricular groove |
| | sinus, at least 5 mm from | pressure of ≧50 mmHg. | site, or adjacent right |
| | any coronary artery that is | Visualize curvilinear line to | or left ventricular |
| | ≧1.0 mm diameter | exit point, judge point at | surface, ≧5 mm |
| | | which cavity should be | from visible coronary |
| | | reached and point at which it | arteries about 75% |
| | | should be exited. Press at | of distance from |
| | | entry point, with preliminary | base to apex |
| 2 [72] | 1.0 cm to one side of LAD, | nick of epicardium if | Posterior inter- |
| | and, if left of LAD, also 1 cm | necessary. After entry into | ventricular groove |
| | below circumflex after | myocardium, press in | site, or adjacent right |
| | clear ultrasound exam for | desired direction and | or left ventricular |
| | intermediate branches and | advance—and monitor | surface, ≧5 mm |
| | perforators. | continued aspiration ease or | from coronary |
| | | oximetric readings | arteries about 75% |
| | | accordingly. Maintain a very | of distance from |
| | | low threshold for withdrawing | base to apex |
| 3 [73] | Conus roof (i.e., the portion | and correcting direction if | Same as #1 exit, but |
| | of the outflow portion of the | chamber not entered or | about 50% of |
| | right ventricle that is | exited at near depth of | distance from base |
| | adjacent the right coronary | penetration expected. Keep | to apex |
| | artery and the right | twist of wrist such as needed | |
| | atrioventricular groove) | to slide ball tip of stylus firmly | |
| | near the tricuspid valve | along septum till opposite | |
| | and between the atrium | angle encountered. Vibratory | |
| | and the right coronary | motion in penetrating out; if | |
| | artery. | vessels of concern in region, | |
| 4 [74] | Conus roof near the | recheck with ultrasound and | Adjacent #2 exit. |
| | pulmonary artery and | possibly redirect. Note | |
| | between the atrium and the | palpable bulge, as well as | |
| | right coronary artery. | visible bulge and blanch, and | |
| 5 | Adjacent exit point for # 3. | then ease out with needle- | Adjacent #4 exit. |
| | | nick in epicardium over exit | |
| | | site. | |

Again, this is a nonlimiting illustration of patterns of placement, from which many deviations in number of stylus paths, their course, or both, are possible. Entrances and exits may be interchanged depending on operative access and visibility. For one example of an alteration, the posterior extreme of path [72], [74], and [75], now adjacent one another, could be separated with that of path [75] changed to a more basal and that of path [74] to a more apical location.

Methods for Placement of Strands for Septal Supporting Net Along Paths of Styli

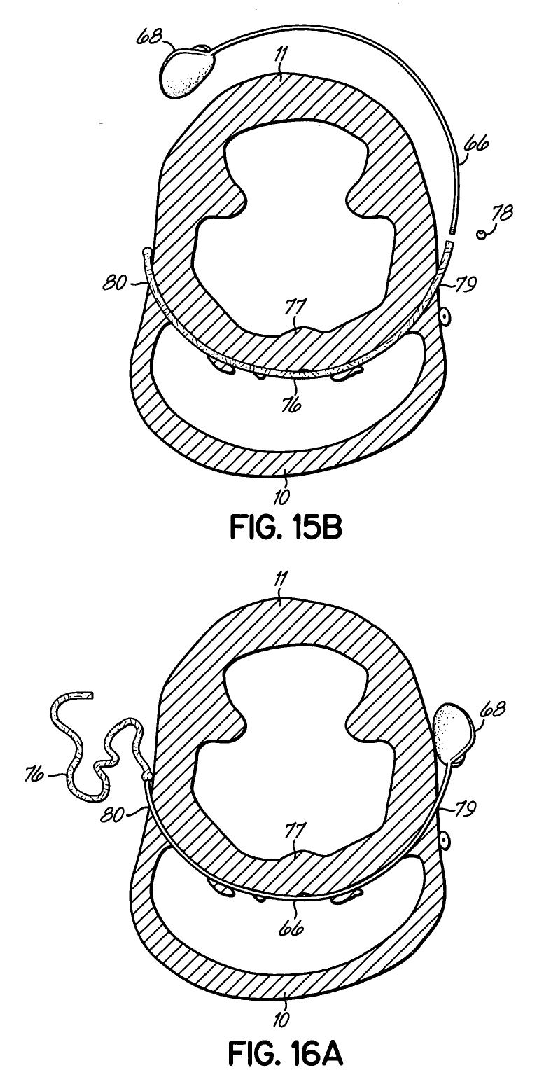

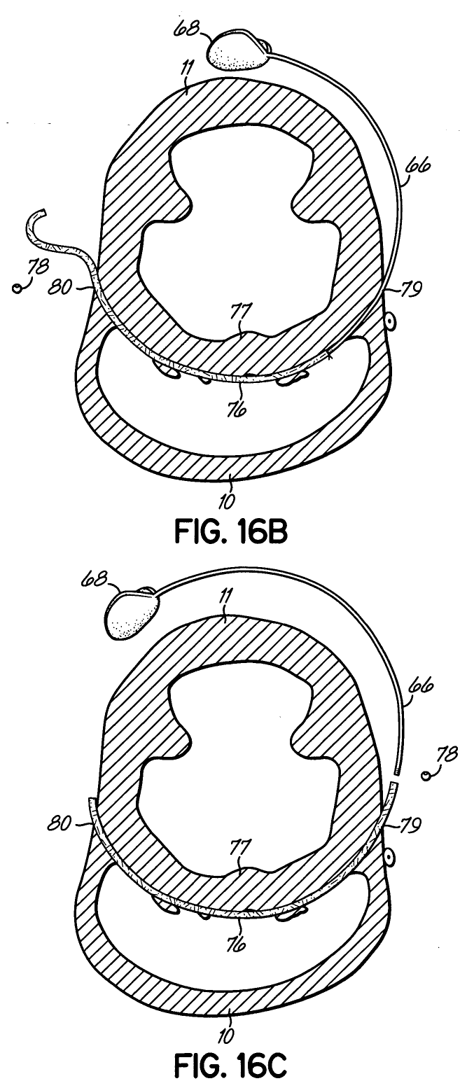

There are two general methods, each derived from commonly used surgical techniques for passing flexible members (e.g., vascular grafts, pacemaker wires, various types of conduits) through closed spaces, and both given as non-limiting examples.
1. (FIGS. 15a and 15b.) The strand is a tubular sleeve [76], and of material such as polyethylene, polypropylene, or polyester mesh or such as expanded PTFE. It is closed at one end, and the opposite end is slipped over the stylus before placement. The stylus [66] is advanced across the right ventricle [10], along the surface of the ventricular septum [77] with the sleeve in place. Upon exit, the tip of the sleeve is grasped with an instrument (such as a forceps or a hemostat, not shown) and the stylus withdrawn, leaving the strand in place. Generally, unless the sleeve is a very open mesh, one or more small openings will be located specifically to permit function of the aspirating port or oximetric sensor. In a slight variation of this method, the ball tip of the stylus is configured to fit on the stylus tip outside, and after placement of, such a sleeve, and to be removed before sleeve grasping and stylus withdrawal.
2. (FIGS. 16a, b and c) The strand [76] is configured to be pulled back through after introduction of the stylus [66]. The tip [67] of the stylus is configured to be attached to the strand after protruding from its exit point. It may be placed over or tied or otherwise fixed to the ball tip of the stylus, or a detachable ball tip [78] may be removed and the strand attached to, for example, a threaded region from which the ball tip had been removed. Attachment may be by tying a suture or other means. One fairly obvious variant would require that the ball tip be threaded and screwed onto the stylus tip, and then unscrewed after exiting. The strand could be then fitted with a threaded adaptor that replaced the ball tip. Regardless of means of attachment, the stylus is withdrawn, leaving the strand traversing the path of the stylus, including initial stylus entrance [79] and exit [80] points.
   Devices and Methods for Securing Heart-Wall Penetrating Strands to Achieve Hemostasis and Protect Myocardial Perfusion

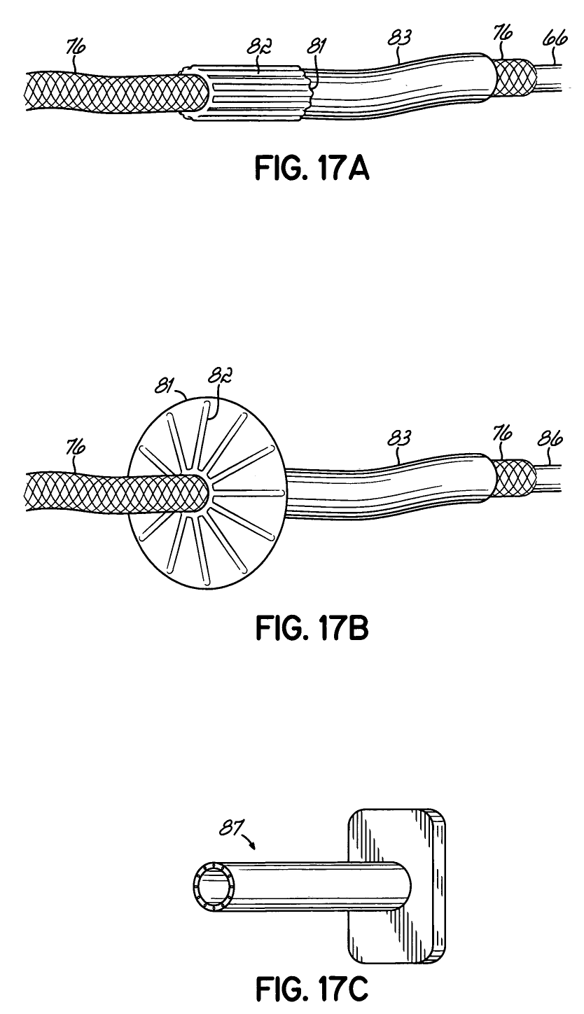

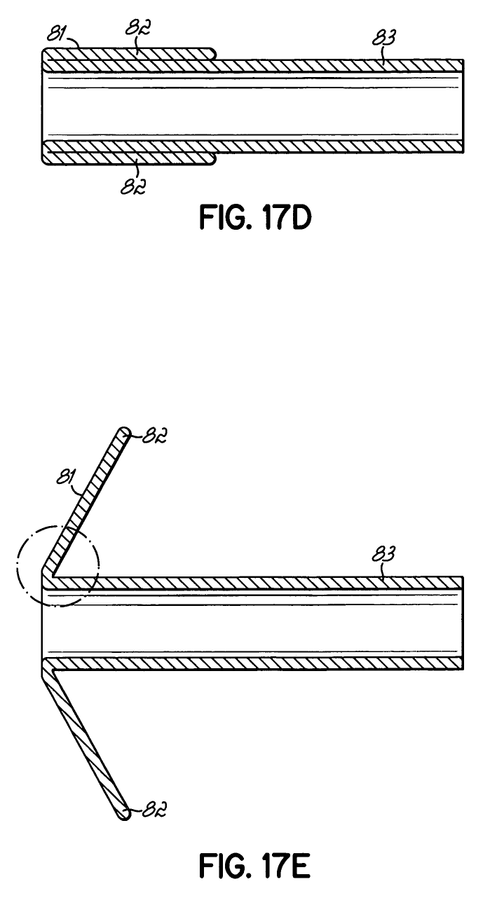

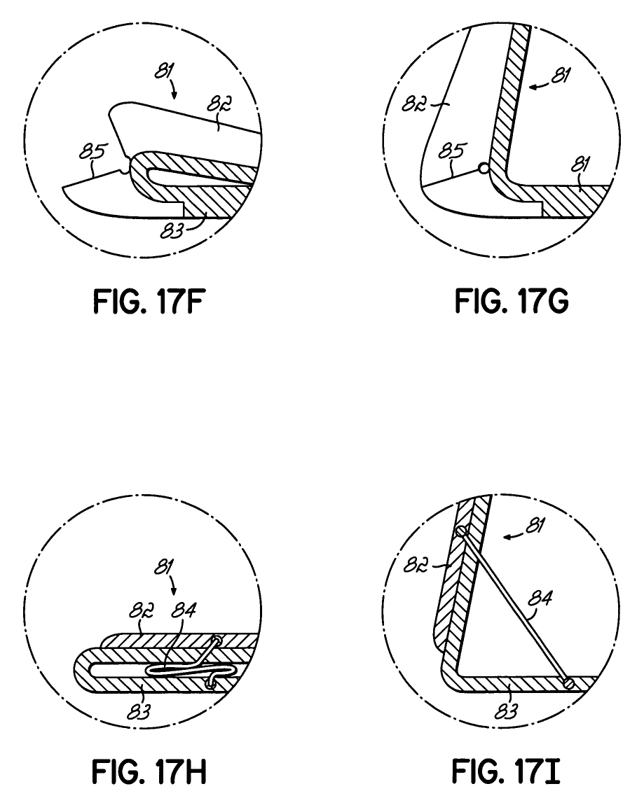

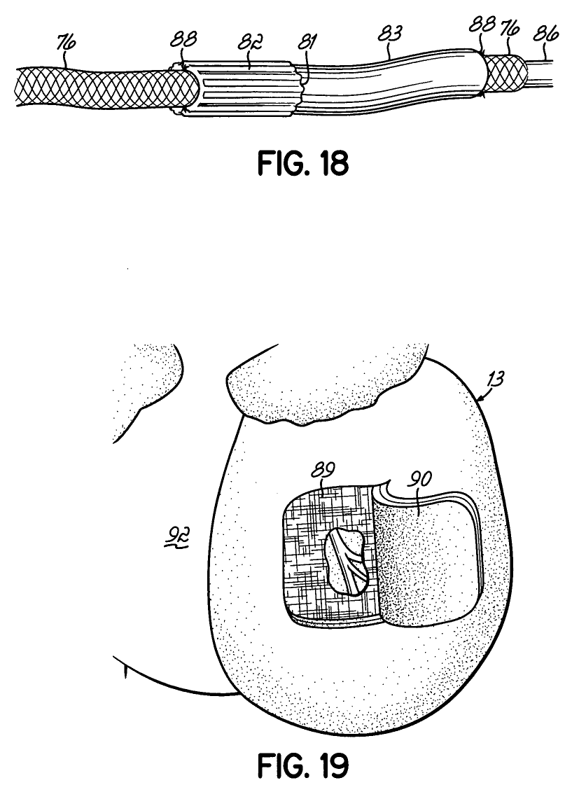

For strands already equipped with or intended to be attached to a bolster that has been placed against the endocardial surface by direct vision, securing by tying or otherwise fixing the external end to either the jacket wall or to a separate button as described above may be sufficient. For strands placed by the methods described immediately above—via punctures, not incisions-special methods may be required.
1. An inner bolster that may be advanced over the strand through the heart wall and expanded in the chamber. One non-limiting example is a balloon, fixed to a tubular stem slipped over the strand and inflated once inside via a small caliber tubing; preferably with a liquid material that solidified into a stable substance (for example, two component epoxy or silicone rubber or polyurethane), with the balloon externally covered by a material such as polyester velour to encourage rapid endothelialization. Another, more preferred but nonlimiting example is an ‘umbrella’ like inner bolster may be advanced over the strand from outside to inside in a closed position and allowed to open in the cardiac chamber, followed by retraction to seat against the inner surface of the cardiac chamber wall. As shown in FIG. 17 a through 17i, this bolster includes a collapsible disc section [81], generally of macro-porous material such as knitted or woven polyester or a micro-porous material such as ePTFE, including several (generally 4 to 8) radially stiff ‘stays’ [82] either of the same material of greater thickness and density or of a separate material and a tubular section [83], generally but not necessarily of the same material as the disc section. The tubular section and disc section would generally be fabricated together in a sort of ‘top hat’ configuration. The tubular section is of adequate internal diameter to fit over the strand, sufficiently stiff to slide over the strand through the heart wall with the disc section collapsed, and sufficiently strong to be pulled back, allowing the disc section to open inside. To limit opening of the disc section (i.e., to prevent over-eversion as tends to occur with umbrellas in high winds), retaining cords [84] may extend from the tubular section [83] to the stays [82] to set an appropriate limit. Alternatively, a ‘stop-ring’ [85] may be affixed to the end of the tubular section at its junction with the disc section, so that stays will impact on it as shown in FIG. 17 e and f). In the event that the strand is tubular, a filler core [86], generally of a solid flexible polymer, may be inserted through the region of wall penetration prior to or following bolster placement to facilitate bolster stability stiffen the traversing strand and tighten its fit inside the tubular section [3]. A pusher tool [87] may be used to push the closed umbrella-like device over the strand through the heart wall; after the disc portion is inside, the pusher tool [87] is withdrawn. Following opening of the disc portion and firm traction on the strand to secure it against the inner heart wall, the external fixation means described above for heart-traversing cords or strands (i.e., independent ‘buttons’ or direct insertion into the margin of an active jacket, securing by means ranging from simple suture tying to tension-controlled mechanical fixation) may be applied.
FIG. 17a is a perspective of the device with disc section [81] collapsed and 17b with it open. FIG. 17c is the pusher tool [81]. FIG. 17d shows a long section of the device closed (above) and open (below). FIGS. 17g and 17h show close-up sectional views of the retaining cord mechanism for preventing eversion and FIGS. 17e and 17f similar views of the stop-ring mechanism.
Alternative means of providing an internal bolstering effect to a strand traversing the heart wall include, in addition to this umbrella-bolster device, means that are similarly derived from the Foley balloon-tipped catheter commonly used to provide self-stabilization in the urinary bladder and derived from the ‘molly-bolt’ commonly used in construction. Means of adaptation may be obvious to those familiar with these fields: fabricating of materials demonstrated to tolerate long-term biologic implantation, providing surfaces that promote tissue ingrowth, and, in the case of the balloon catheter, injecting the balloon not with saline or water, but rather with a solidifying material, either hard (such as epoxy) or soft (such as a silicone rubber or a polyurethane or other biocompatible elastomer).
2. (FIG. 18) A means similar to either of the above examples but allowing firm fixation of inner bolster to strand prior to sliding through the heart wall. To illustrate, in the preferred ‘umbrella’ embodiment, the disc and tubular sections are attached to the strand adjacent the external heart surface, with the heart compressed and the strand under traction so that the region of strand normally at the inner surface is outside the heart. The disk or adjacent tubular portion may be sutured [88] or otherwise securely fixed to the strand, with or without a filler core such as noted above. Then the heart wall is allowed to return to or past normal position, facilitated by pushing on the end of the strand (which may be regionally stiffened by a filler core), such that the disc portion is well in the heart chamber. Traction then opens the disc portion and approximates it to the inner heart chamber surface. External fixation and securing means may then be employed. [part numbers same as 17].
   Surgical Approaches for Placement of the System at Operation Two Methods for Placement of the System at Operation Have Been Developed.

First is a method in which the heart is removed similarly to the technique for removing a donor heart for transplant, has atria separated and right ventriculotomies made, and then is fitted with annuloplasty ring (s), atrial collar(s), great artery sleeve(s), septal rim bolsters, left-side jacket/actuator (if left-only or biventricular system) or, right-side jacket/actuator (unless left-only system), and septal splint, and then reimplanted following closure of the ventriculotomies

Second is a method in which the heart is left in site with atrial separation performed through an approach similar to that used for open mitral valve access, bolsters placed through an approach similar to that used for ventricular septal defect repair, annuloplasty ring placed by standard left atriotomy, jacket rim by ‘parachute suture’ and sequentially lowering and tying similarly to valvular prosthesis placement, collar sleeve and jacket base by separating and reattaching about intact structures, and finally suture closure of atriotomy and ventriculotomies.

Devices and Methods to Facilitate Reoperation

Means have been developed for separation of jacket from heart wall for re-operation, particularly for coronary artery procedures and to protect coronary arteries and conduits after reassembly and reactivation of the active jacket.

The interface between the jacket and the heart surface may be configured to allow removal at subsequent operations for coronary artery disease or other purpose.

The interface of may be achieved by an inner layer [89] that is configured to encourage tissue ingrowth and biologic adhesion, such as polyester velour, and an easily separable bonding [90] of that layer to the remainder of the jacket [13]. (FIG. 19).

The easily separable bonding may be achieved by a hook and loop interface similar to the mechanism of ‘Velcro’. The easily separable bonding may be achieved by low strength polymer adhesive. Sharp dissection of a heart-adhering layer and the remainder of the jacket may be facilitated by contrasting colors. For a non-limiting example, the heart-adhering layer may be white and the facing portions of the remainder of the jacket a bright blue. Metallic components of the jacket may also be incorporated to establish a clear plane of dissection. This helps to guide a sharp dissection, facilitating removal of essentially the full thickness of at least selected regions of a jacket, leaving only a soft polymer fabric adherent to the heart. A non-limiting example is a thin titanium mesh.

The jacket may be fabricated in several modules of which one or more may be removed for access to coronary arteries or other otherwise obscured parts of cardiac anatomy at re-operation, following by replacement and secure mechanical fixation after the intervention.

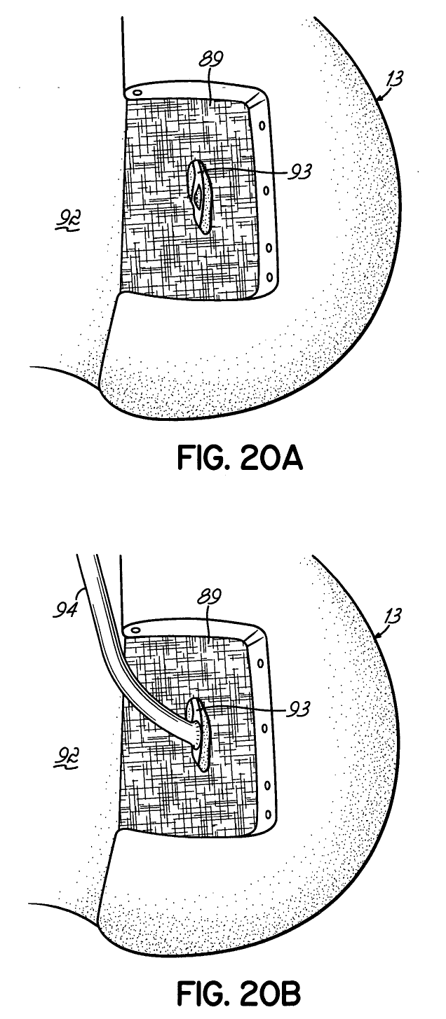

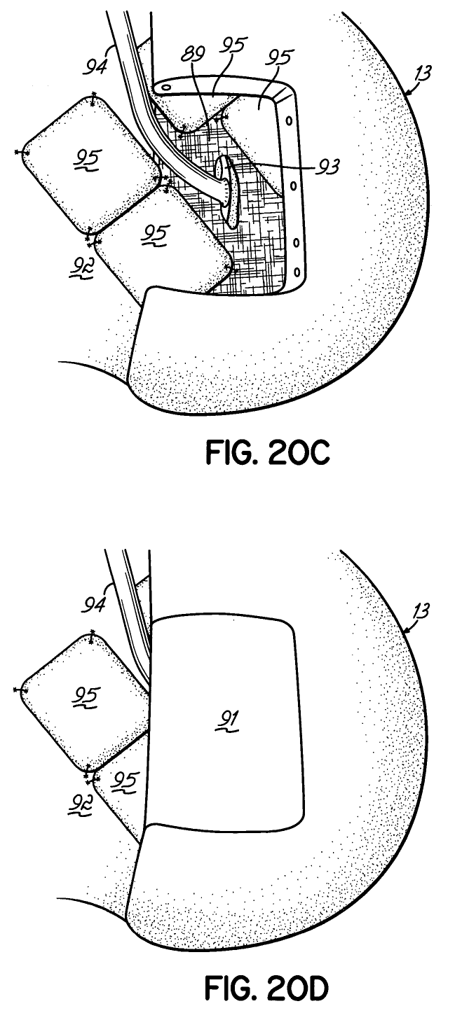

The method of coronary artery intervention in the presence of an adherent mesh after jacket removal generally includes localization and assessment of native coronary arteries through direct surface ultrasonic imaging using techniques familiar in cardiac surgery. A gel-filled or bead-filled cushion may be fabricated in an assortment of sizes and shapes to be placed around, over, or beside a coronary artery conduit, such as an internal thoracic graft, to protect it in the manner of naturally occurring coronary fat pads. These protect the conduit from compression and abrasion after replacement and activation of the jacket. (FIG. 20a through 20e). In 20a a section [91] of a jacket [13] has been removed, leaving a thin polymer mesh [89] adherent to the heart [92], a coronary artery [93] has been localized, preferably by surface imaging, such as with ultrasound, and the mesh incised to see and open the coronary. In 20b a bypass graft [94] has been performed. In 20c preformed, preferably lozenge-shaped, cushions [95] have been selected and placed on either side of the bypass conduit, tacked to the adherent mesh. Alternatively, readily available materials such as Teflon® felt may be cut to form a cushion. In 20d the removed section has been replaced—either by the original piece [91] or by a modular new section. The entire jacket may be removed and replaced using similar techniques if the extent of access required for revascularization or other reason requires that.

While the present invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of applicant's general inventive concept.

What is claimed:

1. A system for assisting in the operation of the natural heart, comprising:
an actuator mechanism configured for positioning at least partially on an outside surface of the heart for deforming the heart;
a structure for securing the actuator mechanism to a natural heart, the structure including a plurality of bolster devices configured for positioning proximate a plurality of points on an inner surface of the heart;
sutures secured to the bolster devices and passing from the bolster devices through the heart wall for being coupled with the actuator mechanism;
the bolster devices anchoring the actuator mechanism to the heart at multiple points along the heart inner surface.

2. The system of claim 1 wherein the bolsters are in the form of beans.

3. The system of claim 2 wherein the bolster is kidney-shaped.

4. The system of claim 2 wherein the bolster is formed of an elastomeric material.

5. The system of claim 2 wherein the bolster is coated with a porous material.

6. The system of claim 1 further comprising a tension element configured to couple with the sutures and set the tension of a suture secured to the bolster device.

7. The system of claim 6 wherein the tension element includes a clip configured to hold the suture at the desired tension.

8. The system of claim 1 wherein the structure for securing the actuator mechanism includes buttons positioned proximate the outer surface of the heart, the sutures spanning between the bolster devices and the buttons.

9. A system for securing an actuator mechanism to an outer surface of a natural heart wherein the actuator mechanism assists in the operation of the natural heart, the system comprising:
a plurality of bolster devices configured for positioning proximate a plurality of points on an inner surface of the heart;
sutures secured to the bolster devices and passing from the bolster devices through the heart wall for being coupled with the actuator mechanism;
the bolster devices anchoring the actuator mechanism to the heart at multiple points along the heart inner surface.

10. The system of claim 9 wherein the bolsters are in the form of beans.

11. The system of claim 10 wherein the bolster is formed of an elastomeric material.

12. The system of claim 9 further comprising buttons positioned proximate the outer surface of the heart, the sutures spanning between the bolster devices and the buttons.

13. A system for mechanically spanning an inner wall of a natural heart to assist in the operation of the heart comprising:
a plurality of strands configured for being positioned across the heart inner wall;
a bolster configured for sliding with respect to a strand from an outer wall of the heart to an inner wall, the bolster including a wall engagement portion which opens inside the heart to engage the heart inner wall and secure the bolster.

14. The system of claim 13 wherein the wall engagement portion includes a balloon structure.

15. The system of claim 13 wherein the wall engagement portion includes a flexible disc that is operable to collapse when the bolster is moved from the outer heart wall to the inner heart wall and to open inside the heart.

16. The system of claim 13 wherein the strands are tubular.

17. The system of claim 16 further comprising cores positionable in the tubular strands to stiffen the strands.

18. A system for assisting in the operation of the natural heart, comprising:
an actuator mechanism configured for positioning at least partially on an outside surface of the heart wherein the actuator mechanism assists in the operation of the natural heart;
a structure for securing the actuator mechanism to a natural heart, the structure including:
a plurality of strands configured for being positioned across a heart inner wall;
a plurality of bolster devices, each configured for sliding with respect to a strand from an outer wall of the heart to an inner wall, the bolster devices including a wall engagement portion that opens inside the heart to engage the heart inner wall and secure the bolster device;
sutures secured to the bolster devices and passing from the bolster devices through the heart wall for being coupled with the actuator mechanism;
the bolster devices anchoring the actuator mechanism to the heart at multiple points along the heart inner surface.

19. The system of claim 18 wherein the wall engagement portion includes a balloon structure.

20. The system of claim 18 wherein the wall engagement portion includes a flexible disc that is operable to collapse when the bolster device is moved from the outer heart wall to the inner heart wall and to open inside the heart.

21. The system of claim 18 wherein the strands are tubular.

22. The system of claim 21 further comprising cores positionable in the tubular strands to stiffen the strands.

23. The system of claim 18 wherein the bolster devices are fixed to the strands.

24. An actuator mechanism configured for positioning at least partially on an outside surface of the heart for deforming the heart to assist in the operation of the natural heart, the actuator mechanism comprising:
a jacket configured to be secured to an outer surface of the heart;
the jacket including:
an inner adhesion layer configured for being coupled with an outer surface of the heart;
an outer operating layer separably coupled with the inner adhesion layer.

25. The actuator mechanism of claim 24 further comprising hook and loop fasteners positioned between the inner adhesion layer and outer operating layer.

26. The actuator mechanism of claim 24 further comprising a low strength adhesive positioned between the inner adhesion layer and outer operating layer.

27. The actuator mechanism of claim 24 wherein the inner adhesion layer and the outer operating layer are different colors.

28. The actuator mechanism of claim 24 further comprising a metallic layer between the inner adhesion layer and the outer operating layer.
